(19) United States
(12) Patent Application Publication
(10) Pub. No.: US 2006/0282166 A1
(43) Pub. Date: Dec. 14, 2006

(54) Compliant porous coating

(75) Inventors: Fred Molz, Collierville, TN (US); Hai H. Trieu, Cordova, TN (US); James E. Van Hoeck, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/148,228

(22) Filed: Jun. 9, 2005

(51) Int. Cl. A61F 2/44; A61F 2/28

(52) U.S. Cl. 623/17.13; 623/23.6; 623/17.11

(57) ABSTRACT

Intervertebral implant components having compliant coatings, and methods of making and implanting the implant components are provided. The embodiments relate to compositions, methods and devices having a compliant surface coating that permits application of the device in areas without significant bone reformation to accept the device.

### FIELD OF THE INVENTION

Embodiments relate to coated devices, methods of making the devices, and methods of using the devices. More specifically, the embodiments relate to methods and devices having a compliant surface coating that permits insertion of the device in areas without significant bone reformation to accept the device.

### DESCRIPTION OF RELATED ART

Many medical devices and implants are designed to contact a bony surface. Some of these devices are provided with coatings to enhance bone growth and integrate the device into the bony tissue. To minimize pain and injury, the bony surface typically is modified, or reformed, to closely match the three-dimensional profile of the medical device.

Other medical devices and implants are designed to articulate with another implant, or with bone. For example, hip replacements typically include a femoral stem with a spherical head, which is capable of articulating within an acetabular cup. The repetitive articulation within the acetabular cup may cause the cup itself to move with respect to the adjacent bone. Moreover, placement of the acetabular cup requires preparation of the bony surface to accept the back surface of the acetabular cup, which in many cases includes a number of perforations or metal projections to secure the cup to the bone.

For example, U.S. Pat. No. 4,769,041 discloses a hip joint socket body that is covered with a multi-layer grid having a plurality of peg-like projections of metal.

The peg-like projections provide an interface to prevent the plastic cup portion from directly contacting the bone. The '041 patent discloses promoting ingrowth and accretion of tissue by providing a coating grid having more than two layers with the pore size of the grid openings increasing outwardly from layer to layer.

U.S. Pat. No. 4,963,154 discloses an acetabular cup as a part of a hip joint prosthesis, comprising an outer support ring and a plastic inner socket. The cup further includes a covering cap having at least one metal surface. The outer support ring includes a number of supporting flanks that are self-tapping threads such that the acetabular cup can be screwed directly into the acetabulum socket without prior cutting. The cap also can be provided with a porous layer (or porocoat) that is pure titanium and is said to enhance growth of the cap into the bone region to provide secondary fixation.

U.S. Pat. Nos. 4,851,004 and 5,222,985, the disclosures of which are incorporated by reference herein in their entirety, disclose an intramedullary prosthesis for a hip prosthesis utilizing a tapered elongate stem that is undersized and coated with a compressible resilient coating. The coating renders the stem somewhat oversized with respect to the void space in the stem socket, and fills the void area between the undersized stem and the precisely formed stem socket.

Some medical devices are coated with materials to prevent contamination. Various medical devices that are inserted into body cavities of humans and animals can unfortunately introduce bacterial, viral and fungal infections into these body cavities. Numerous coatings are available for medical devices that employ polyurethane or urethane pre-polymers to act as lubricants, drug delivery systems and the like. Known coatings applied to surfaces of medical devices include coatings of polyvinylpyrrolidone, polyurethane, acrylic polyester, vinyl resin, fluorocarbons, silicone rubber, and combinations of these substances. For example, U.S. Pat. Nos. 4,100,309 and 4,119,094 to Micklus et al., relate to a hydrophilic coating of polyvinylpyrrolidone-polyurethane interpolymer formed using polyisocyanate. To prevent infections, various anti-microbial methods and compositions have been disclosed in U.S. Pat. Nos. 4,054,139; 4,592,920 and 4,603,152. Additionally, U.S. Pat. No. 3,939,049 to Ratner et al. relate to a method of grafting hydrogels (for lubrication) to polymeric substrates using radiation, U.S. Pat. No. 3,975,350 to Hudgin et al. relate to hydrophilic polyurethane polymers for use as lubricants, and U.S. Pat. No. 3,987,497 to Stoy et al. relate to a tendon prosthesis having a lubricant hydrogel coating.

The art is rife with disclosures of implants coated with bone growth promoting coatings, such as hydroxyapatite coatings. These coatings are said to enhance bone growth between the implant and the adjacent bony structures, thereby enhancing the implants' fixation in the bone. Representative disclosures include, for example, U.S. Pat. Nos. 4,177,524, 5,021,062, 5,071,437, 5,658,285, 5,716,359, 6,008,431, 6,102,948, 6,572,653, 6,582,468, 6,699,288, 6,736,849, 6,743,256, and 6,790,233, the disclosures of which are incorporated herein by reference in their entirety.

While these coatings may be sufficient for promoting or enhancing bony growth, they are hard coatings that do not permit the implant surface to conform to the adjacent bone surface. Consequently, the surgeon typically must prepare the bony surface to accept the implant, depending on the shape of the implant.

Grinding of bone to accept an implant is an especially delicate task when performing spine surgery, particularly spine fusion surgery. Disc replacement devices or spinal implants are configured to be load bearing bodies of a size to be placed in an intervertebral disc space, and they are intended to fully or partially replace the nucleus pulposus of mammals, particularly humans. Prior to implantation, the nucleus pulposus is removed, and the endplates of the adjacent vertebral bodies are shaped to accept the implant. Shaping of the endplates is time consuming and intricate, and it sometimes removes load-bearing portions of the vertebral body. Techniques for preparing the bony surfaces of vertebral endplates to accept an intervertebral prosthetic disc are described in, for example, U.S. Pat. Nos. 6,083,228; 6,517,544; and 6,537,279; and U.S. patent application Publication Nos.: 2002/0035400; 2002/0128715; 2002/0151901; 2003/0187448; 2003/0130662; and 2005/0015091, the disclosures of which are incorporated by reference herein in their entirety.

Certain areas on the vertebral endplates carry more load than other areas, and consequently, the disc in that area must bear additional load. Most replacement discs are designed to mirror as closely as possible the vertebral endplates, but some machining typically is required, either of the device itself, or of the endplates just prior to implantation. Hard coatings on the surface of the device fail to account for the differences in load bearing characteristics across the surface area of the endplates, and they typically are milled (or the bone surface is milled) prior to implantation.

U.S. Pat. No. 6,863,689 discloses an intervertebral spacer having a flexible wire mesh welded thereto. The convex (or domed) wire mesh is said to deflect as necessary during implantation, and once seated between the vertebral bodies, deforms as necessary under anatomical loads to reshape itself to the concave surface of the vertebral endplate. The spacer described therein facilitates fusion of the two vertebral bodies, and is not intended to be used as a prosthetic disc preserving motion between the vertebral bodies.

The description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

A need exists for a device and method to provide a more readily adaptable medical implant that contacts a bony surface. A need also exists for a device and method to impart conformable surfaces to otherwise rigid bodies enabling their implantation and more intimate contact with non planar bony surfaces. A need also exists for a device and method that provides variable coating compliance across its cross-section thereby enabling certain parts to be more compliant than others. A need also exists for an intervertebral prosthesis that can be more easily implanted into an at least partially evacuated disc space, without the need for substantial or any vertebral endplate machining prior to implantation.

A feature of an embodiment of the invention therefore provides devices and methods of making and using the devices, whereby the devices contain a coating on a substrate, the coating being compliant and capable of deformation. An additional feature of an embodiment of the invention provides devices and methods of making and using the devices, where the devices contain a compliant coating rendering the device suitable for implantation adjacent a non-planar and/or non-uniform bony surface. An additional feature of an embodiment of the invention provides devices and methods of making or using the devices, where the devices have compliant coatings that vary in deformability across the surface of the device.

Another feature of an embodiment provides devices and methods of making or using the devices, where the devices have a coating that provides for improved bony ingrowth. These and other features are satisfied by the embodiments described herein.

In one embodiment, there is provided an intervertebral disc prosthesis and/or prosthesis component having a compliant coating on at least one surface, the at least one surface intended to contact a vertebral bone in a mammal's body, the compliant coating at least partially conforming to the vertebral bone. Such an intervertebral disc prosthesis permits implantation without excessive (and preferably without any) vertebral body milling to conform the bone surface to fit the prosthesis.

An additional embodiment provides a method of making an intervertebral disc prosthesis component comprising forming a disc prosthesis component, and coating at least one bone-contacting surface of the prosthesis component with a coating material that, when coated on the surface, provides a compliant surface capable of deformation and partial conformation to a vertebral bone in a mammal's body.

Another embodiment provides a method of inserting an intervertebral disc prosthesis into an at least partially evacuated disc space comprising partially evacuating the disc space; providing an intervertebral disc prosthesis having at least one component with a compliant coating on at least one surface, the at least one surface intended to contact a vertebral bone in a mammal's body, the compliant coating at least partially conforming to the vertebral bone. The method further includes inserting the prosthesis into the at least partially evacuated disc space without the need for significant (an preferably without any) modification of at least one of the vertebral bony surface prior to insertion.

These and other features and advantages of the embodiments will be apparent from the description provide herein.

### BRIEF DESCRIPTION OF THE FIGURES

FIG. 1, embodiments A and B, is an illustration of the CHARITÉ™Artificial Disc and its implantation between two vertebral bodies showing gaps between the disc and the bone.

FIG. 2 illustrates a side view of an intervertebral prosthesis component having a compliant coating on a bone-contacting surface.

FIG. 3 illustrates a side view of another intervertebral prosthesis component having a compliant coating on a bone-contacting surface.

FIG. 4 illustrates a side view of a cross-section of an intervertebral prosthesis showing a variable conformability in the compliant coating to correspond to high load areas of the prosthesis.

FIG. 5, embodiments A, B, and C illustrate an intervertebral prosthesis component conforming to the bone surfaces of an vertebral body upon insertion, due to the presence of a compliant coating.

### DESCRIPTION OF THE PREFERRED EMBODIMENTS

The following description is intended to convey a thorough understanding of the present invention by providing a number of specific embodiments and details involving intervertebral disc prosthesis, methods of their manufacture, and methods of their use. It is understood, however, that the present invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

The terminology used herein is for the purpose of describing particular embodiments only and is not intended to limit the scope of the present invention.

As used throughout this disclosure, the singular forms “a,” “an,” and “the” include plural reference unless the context clearly dictates otherwise. Thus, for example, a reference to “a bone-contacting surface” includes a plurality of such surfaces, as well as a single surface, and a reference to “an intervertebral disc prosthesis component” is a reference to one or more components and equivalents thereof known to those skilled in the art, and so forth.

Unless defined otherwise, all technical and scientific terms used herein have the same meanings as commonly understood by one of ordinary skill in the art to which this invention belongs. All publications mentioned herein are cited for the purpose of describing and disclosing the various implants, prosthesis, components, methods of implantation, coatings and surface treatments, and other components that are reported in the publications that might be used in connection with the embodiments. Nothing herein is to be construed as an admission that the embodiments described herein are not entitled to antedate such disclosures by virtue of prior invention.

Throughout this description, the expressions “intervertebral disc prosthesis” or “intervertebral disc prosthesis component” shall be used to denote any man-made implant material or component thereof that is used to partially or fully replace the natural nucleus pulposus or intervertebral disc that is found in mammals, especially humans. Man-made intervertebral disc prosthesis include prosthesis made from natural sources (e.g. implanted autologous bones and tissues), implants made from synthetic sources (e.g. metals, polymers, and ceramics), and composites thereof (e.g. bone/polymer matrices, metal/polymer composites, and the like).

Intervertebral disc prosthesis and components thereof can be made using a wide range of materials such as polymeric materials, metals, ceramics, and body tissues.

Exemplary polymeric materials include, but are not limited to, thermoplastic polymers, thermoset polymers, elastomers, hydrogels, adhesives, sealants, and composites thereof. Preformed disc prosthesis implants may be in any shape, including implants shaped like a spiral, hockey puck, kidney, capsule, rectangular block, cylinder, implants such as those described in, for example, U.S. Pat. No. 6,620,196, the disclosure of which is incorporated herein by reference in its entirety, and the like. Disc prosthesis, especially polymeric implants, also may comprise supporting bands or jackets.

Intervertebral disc prosthesis, and components thereof, may be in any of numerous known forms, including, but not limited to, total disc prostheses, intervertebral fusion devices, stackable corpectomy devices, threaded fusion cages, impacted fusion cages, end plates, screws, outer sheaths or bags, etc. Intervertebral disc prosthesis also include implants wherein only the full or partial nucleus of the intervertebral disc is replaced, for example nucleus replacement implants and nucleus augmentation implants. Preferably, the intervertebral disc prosthesis is a total disc prosthesis or partial disc prosthesis intended to preserve motion between the vertebral bodies, and is not a fusion device.

Exemplary intervertebral disc prosthesis that can benefit from the compliant coatings described in the embodiments herein include, but are not limited to, those described in U.S. Pat. Nos.: 5,002,576; 5,071,437; 6,348,071; 5,146,933; 5,514,180; 5,458,643; 5,522,898; 5,705,780; 5,676,702; 5,370,697; 5,320,644; 4,863,477; 4,932,969; 4,874,389; 6,132,465; 6,136,031; 6,296,664; 6,306,177; 3,867,782; 4,911,718; 5,171,281; 5,545,229; 5,824,094; 6,113,640; 6,093,205; 5,964,807; 5,258,031; 5,314,477; 5,676,701; 5,425,773; 5,306,308; 5,683,465; 5,899,941; 6,019792; 6,179874; 6,063,121; 6,113,637; 6,048,342; 5,674,296; 5,865,846; 6,001,130; 6,156,067; 5,556,431; 5,401,269; 5,888,226; 6,146,421; 6,228,118; 4,759,769; 5,458,642, the disclosures of each of which are incorporated by reference herein in their entirety. Any type of intervertebral disc prosthesis, or component thereof, disclosed in these documents can be processed in accordance with the embodiments described herein, to include a coating on at least one its surfaces that is intended to contact a bony portion of a vertebral body.

“Disc space” means the volume occupied, or formerly occupied, by the nucleus pulposus. The disc space may be the volume contained inside the annulus fibrosis.

The disc space also may be the entire volume, including the annulus fibrosis, between two adjacent vertebral bodies.

The intervertebral disc prosthesis components that may contain the compliant coatings described herein include any component that is intended to contact a bony surface of a vertebral body. Suitable components that may be coated include prosthesis end plates or hard surfaces that surround or otherwise contain a softer, flexible portion capable of preserving motion. Other components include plates used to attach endplate components to adjacent vertebral bodies, spikes or serrated ridges used to provide better contact between prosthesis end plates and the bony surfaces, and flexible or relatively inflexible polymeric materials that form all or only a portion of the intervertebral prosthesis.

The compliant coating can be comprised of any material or combinations of materials that are conformable, or that can be made to be conformable (e.g., by application of heat, light, pressure, water, etc.), and that are capable of adhering to a surface of an intervertebral disc prosthesis component. For example, the compliant coating may be comprised of a thermoplastic material that is relatively non-compliant at body temperatures, but that is compliant and “moldable” upon application of an appropriate amount of heat (e.g., like a thermoplastic polymer). Alternatively, the compliant coating may be comprised of a curable-type composition that initially has waxy and deformable characteristics, but that cures upon application of fluid, time, body heat, etc., to form a substantially less deformable characteristic after implantation. Compositions similar to those used in forming molds for dental prosthesis, such as crowns, bridges, and the like, can be used as all or a portion of the compliant coating. These known coatings (as well as the known coatings described below) would be modified as described in the embodiments to include other agents described herein, as well as to provide the requisite attachment to the particular material to which the coating is applied.

It also is preferred that the compliant coating be somewhat porous or porous and that it contain or cover another coating that contains bone growth promoting materials. It also is preferred that the compliant coating provide a network or scaffolding to facilitate new bone growth to securely attach the prosthesis component to the bone. Preferably, the compliant porous coating's interconnected pore size is selected to enable cell penetration and tissue ingrowth, and more preferably, the pore size is within the range of from about 100 to about 500 microns. It also is preferred that the compliant coating be capable of soaking or absorbing fluids and binding proteins such as BMP or other growth factors. The expression “compliant coating” is intended to exclude wire mesh materials, such as those described in, for example, U.S. Pat. No. 6,863,689.

Suitable compliant coating materials include, for example, polymeric materials, organic materials, biologic materials, resorbable materials, semi-resorbable materials; synthetic materials, foams, waxes, and other compliant materials capable of being formed into a relatively thin film on an intervertebral disc prosthesis component. Representative components of a compliant coating may include resorbable fibers, woven or interconnected fibers, intepenetrating network fibers (metallic or polymeric), three-dimensional polyethylene, polyesters, PEEK films, titanium mesh fibers, nitinol fibers, PET, PTFE fibers, graphite fibers, polysulfones, hydrogels, flexible tissue scaffolding, resilient film-forming olefinic resins typically used in paints to resist chipping, waxes, biologically grown materials (e.g., collagen, cartilagenous tissue, etc.), and mixtures thereof.

One useful compliant coating composition is described in U.S. Pat. No. 3,992,725, the disclosure of which is incorporated by reference herein in its entirety. In its preferred form, the compliant coating may comprise a resilient, fibrous porous structure comprised of carbon or graphite fibers, optionally in admixture with a proportion of polytetrafluoroethylene fibers, bonded together with a sintered polytetrafluoroethylene resin.

The compliant coating referred to herein desirably is polymeric in nature and preferably is bioresorbable. By compliant, we refer to the ability of the material to be deformed when placed under stress without exhibiting brittle failure, the deformation tending to distribute stress within the article. Preferably, the polymeric material is a bioresorbable polymer that may be one or a combination of:

collagen, poly (lactic acid), poly (glycolic acid), copolymers of lactic acid and glycolic acid, chitin, chitosan, gelatin, or any other resorbable polymer. This polymer material may be used alone, may be reinforced with a particulate or fibrous biocompatible material, and may include one or more biological agents capable of inducing bone formation. Collagen and other polymeric materials may serve as suitable carriers of osteoinductive materials such as BMP and various bone growth proteins, some of which are discussed briefly below. Bioresorbable polymeric materials are preferred coating materials because they are believed to resorb as host bone grows into the interstices to replace it.

Other suitable coating compositions include those described in, for example, U.S. Pat. No. 6,309,660, the disclosure of which is incorporated by reference herein in its entirety. Such resilient coatings preferably include a water soluble polymer layer ionically bound with a second water soluble polymer forming an insoluble molecular film. This molecular film is an electrolyte complex which is further stabilized by the addition of a second layer containing a mixture of at least one multi-functional polymer (a polymer having two or more reactive groups), at least one crosslinking agent reactive with the multi-functional polymer(s), and optionally, one or more useful biologically active compound(s). The multi-functional polymer(s) and crosslinkers(s) of the second layer form an interpenetrating network (IPN) that entraps all biologically active compound(s) added to the mixture and the polymers of the molecular film. Other IPNs made from any of the previously described materials can be used in the embodiments described herein.

The bioactive coatings disclosed in U.S. Pat. No. 5,876,454, the disclosure of which is incorporated by reference herein in its entirety, also may be used in whole or in part in the embodiments described herein. The resilient coating may be comprised of a bioactive conjugate adapted to coat a metal implant outer surface, the bioactive conjugate being comprised of the following structural formula I:
—R—X—PI
wherein, R is O or S, adapted to be covalently attached to an implant surface;

I is the implant surface;

X is selected from a bond, linear or branched chains of 1 to 30 covalently attached atoms selected from the group consisting of C, N, O, Si or S or other linking atoms, rings of 1 to 20 covalently attached atoms selected from the group consisting of C, N, O, Si or S or other linking atoms and a combination of rings and chains of similar composition; and

P is a covalently-attached bioactive molecule moiety which promotes tissue growth, stabilization and integration, and wherein the moiety retains its biological activity.

The compliant coating also may be comprised of collodion. Throughout this description, the term “collodion” denotes any of a group of colorless or pale-yellow, viscous solutions of pyroxylin or nitrocellulose in a mixture of alcohol and ether, which dries quickly and forms a tough, elastic film. The collodion coating then preferably is made porous by inclusion of pore formers or mechanical etching to provide a porous coating.

Other useful coating compositions are comprised of resilient polymeric materials, such as polyethylene/polypropylene impact copolymers, resilient mesh materials, polyurethane foam materials. The coating composition also may have properties similar to a foam material, and be comprised of natural or synthetic materials. The foam material is deformable and porous thereby enabling good bony contact after implantation, and a scaffolding surface to promote bone growth.

The compliant coating described in the embodiments also can be prepared by growing a coating on an intervertebral prosthesis component. Growth of biological coatings are known in the art, and can include growing a controlled biologic coating (e.g., cartilage-like) that undergoes calcification during normal healing and when stressed. The growth can be controlled to provide the appropriate geometry of the coating for the individual patient, and to provide selective areas on the surface of the component that are most likely to be subjected to increased load.

Skilled artisans also are capable of pre-stressing the intervertebral prosthesis component during growth of the biologic coating to control the orientation and structure of the coating.

Biologically grown materials can be loaded or stressed to stimulate growth of tissue in certain areas having an appropriate orientation and structure. For example, in areas where high loads are anticipated, biological growth can be stimulated to a greater degree than other areas, or the tissue can be stimulated to grow in an orientation that renders it more capable of handling higher loads in those select areas.

The compliant coating compositions preferably provide a porous coating that promotes bony ingrowth and secure attachment of the intervertebral prosthesis component and the adjacent bone. Bone growth promoting materials may be combined with the compliant coating composition, or may be coated onto the intervertebral prosthesis component surface and consequently, be positioned between the component surface and the compliant coating. Any configuration is suitable in the embodiments described herein, but it is particularly preferred to provide a coating comprised of two layers: (i) a relatively non-porous, bone growth promoting substance-containing inner layer adjacent the prosthesis component surface; and (ii) a compliant, compressible porous outer layer on the other side of the inner layer from the prosthesis component surface.

Any known bone growth promoting substance can be used in the embodiments described herein. The bone growth promoting substance can be applied as a separate coating beneath the porous, compliant coating, or can be included in the porous, compliant coating. Metallic implant surfaces are commonly coated with micro-porous ceramics such as hydroxyapatite (HA) or beta-tricalcium phosphate (TCP), see U.S. Pat. Nos. 4,309,488; 4,145,764; 4,483,678; 4,960,646; 4,846,837, the disclosures of which are incorporated by reference herein in their entirety. The former treatment is more common because calcium-phosphate salts tend to be absorbed, in vitro, and thus loose their effectiveness. The HA coatings increase the mean interface strength of titanium implants as compared to uncoated implants (see Cook et al., Clin. Ortho. Rel. Res., 232, p. 225, 1988). In addition, clinical trials in patients with hip prosthesis have demonstrated rapid bone growth on prosthetic devices and increased osteointegration of titanium alloy implants when coated with HA (see Sakkers et. al., J. Biomed. Mater. Res., 26, p. 265, 1997).

The HA ceramic coatings can be applied with a plasma spray machine or by sintering (see U.S. Pat. No. 4,960,646). In addition, the HA coating can be applied by soaking the implant in an alkali solution that contains calcium and phosphorous and then heated to deposit a film of hyroxylapetite (see U.S. Pat. 5,609,633).

Optimal HA coating thickness ranges from 50-100 microns (see Thomas, Orthopedics, 17, p. 267-278, 1994). If coated too thick the interface between the HA and bone becomes brittle.

Bone growth-promoting formulations useful for promoting the in-growth and on-growth of endogenous tissues may comprise bone morphogenetic factors. Bone morphogenetic factors are growth factors whose activity is specific to bone tissue including, but not limited to, demineralized bone matrix (DBM), bone protein (BP), bone morphogenetic protein (BMP), and mixtures and combinations thereof. Methods for producing DBM are well known in the art, and DBM may be obtained following the teachings of O'Leary et al. (U.S. Pat. No. 5,073,373) or by obtaining commercially available DBM formulations such as, for example, AlloGro® (commercially available from AlloSource, Centennial, Colorado). Additionally, formulations for promoting the in-growth and on-growth of endogenous bone may comprise bone marrow aspirate, bone marrow concentrate, and mixtures and combinations thereof. Methods of obtaining bone marrow aspirates as well as devices facilitating extraction of bone marrow aspirate are well known in the art and are described, for example, by Turkel et al. in U.S. Pat. No. 5,257,632.

The bone-growth-promoting formulations (or compliant coatings, if different layers) of the embodiments described herein optionally may further comprise antibiotics and antiretroviral drugs. As discussed by Vehmeyer et al., the possibility exists that bacterial contamination can occur, for example, due to the introduction of contaminated allograft tissue from living donors. Vehmeyer, SB, et al., Acta Orthop Scand., 73(2):165-169 (2002). Antibiotics and antiretroviral drugs may be administered to prevent infection by pathogens that are introduced to the patient during implant surgery. Also, administration of antibiotics and antiretroviral drugs may be useful to account for nosocomial infections or other factors specific to the location where the implant surgery is conducted. Antibiotics and antiretroviral drugs include, but are not limited to, aminoglycosides such as tobramycin, amoxicillin, ampicillin, azactam, bacitracin, beta-lactamases, beta-lactam (glycopeptide), biomycin, clindamycin, chloramphenicol, chloromycetin, cefazolin, cephalosporins, ciprofloxacin, erythromycin, fluoroquinolones, gentamicin, macrolides, metronidazole, neomycin, penicillins, polymycin B, quinolones, rapamycin, rifampin, streptomycin, sulfonamide, tetracyclines, trimethoprim, trimethoprim-sulfamethoxazole, vancomycin, and mixtures and combinations thereof.

The bone growth-promoting formulations (or compliant coatings, if different layers) of the embodiments described herein optionally may further comprise immunosuppressive agents, particularly in circumstances where an implant comprising an allograft composition is delivered to the patient. Suitable immunosuppressive agents that may be administered include, but are not limited to, steroids, cyclosporine, cyclosporine analogs, cyclophosphamide, methylprednisone, prednisone, azathioprine, FK-506, 15-deoxyspergualin, and other immunosuppressive agents that act by suppressing the function of responding T cells. Other immunosuppressive agents include, but are not limited to, prednisolone, methotrexate, thalidomide, methoxsalen, rapamycin, leflunomide, mizoribine (bredininTM), brequinar, deoxyspergualin, and azaspirane (SKF 105685), Orthoclone OKTTM 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrexate), Oxsoralen-Ultra™ (methoxsalen) and Rapamuen™ (sirolimus).

The bone growth-promoting formulations (or compliant coatings, if different layers) of the embodiments described herein optionally may comprise substances that enhance isotonicity and chemical stability. Such materials are non-toxic to patients at the dosages and concentrations employed, and include buffers such as phosphate, citrate, succinate, acetic acid, and other organic acids or their salts; antioxidants such as ascorbic acid; low molecular weight (less than about ten residues) polypeptides such as polyarginine and tripeptides; proteins such as serumalbumin, gelatin, and immunoglobulins; amino acids such as glycine, glutamic acid, aspartic acid, and arginine; monosaccharides, disaccharides, and other carbohydrates including cellulose and its derivatives, glucose, mannose, and dextrans; chelating agents such as EDTA; sugaralcohols such as mannitol and sorbitol; counterions such as sodium; nonionicsurfactants such as polysorbates, poloxamers, and polyethylene glycol PEG; and mixtures and combinations thereof.

The bone growth-promoting formulations may comprise osteoinductive and osteoconductive agents. Such agents include, but are not limited to members of the families of Bone Morphogenetic Proteins (BMPs), Osteoprotegerin or any of the other osteoclastogenesis inhibitors, Connective Tissue Growth Factors (CTGFs), Vascular Endothelial Growth Factors (VEGFs), Transforming Growth Factor-betas (TGF-bs), Growth Differentiation Factors (GDFs), Cartilage Derived Morphogenic Proteins (CDMPs), and Lim Mineralization Proteins (LMPs).

BMPs are a class of proteins thought to have osteoinductive or growth-promoting activities on endogenous bone tissue, or function as pro-collagen precursors.

Known members of the BMP family that may be utilized as osteoinductive agents in tissue in-growth and on-growth formulations of the invention include, but are not limited to, BMP-1, BMP-2, BMP-3, BMP-4, BMP-5, BMP-6, BMP-7, BMP-8, BMP-9, BMP-10, BMP-11, BMP-12, BMP-13, BMP-15, BMP-16, BMP-17, and BMP-18 polynucleotides and polypeptides, as well as mature polypeptides and polynucleotides encoding the same. The BMPs may be included in the coatings as full length BMPs or fragments thereof, or combinations or mixtures thereof, or as polypeptides or polynucleotides encoding the polypeptide fragments of all of the recited BMPs.

Osteoclastogenesis inhibitors inhibit bone resorption by osteoclasts of the bone tissue surrounding the site of implantation. Osteoclast and Osteoclastogenesis inhibitors include, but are not limited to, Osteoprotegerin polynucleotides and polypeptides, as well as mature Osteoprotegerin polypeptides and polynucleotides encoding the same. The Osteoprotegerin protein specifically binds to its ligand, osteoprotegerin ligand (TNFSF11/OPGL), both of which are key extracellular regulators of osteoclast development. Osteoclastogenesis inhibitors further include, but are not limited to, chemical compounds such as bisphosphonate, 5-lipoxygenase inhibitors such as those described in U.S. Pat. Nos. 5,534,524 and 6,455,541 (herein incorporated by reference), heterocyclic compounds such as those described in U.S. Pat. No. 5,658,935 (herein incorporated by reference), 2,4-dioxoimidazolidine and imidazolidine derivative compounds such as those described in U.S. Pat. Nos. 5,397,796 and 5,554,594 (herein incorporated by reference), sulfonamide derivatives such as those described in U.S. Pat. No. 6,313,119 (herein incorporated by reference), and acylguanidine compounds such as those described in U.S. Pat. No. 6,492,356 (herein incorporated by reference).

CTGFs are a class of proteins thought to have growth-promoting activities on connective tissues. Known members of the CTGF family include, but are not limited to, CTGF-1, CTGF-2, and CTGF-4, any of which may be incorporated into the coating composition of the embodiments, in addition to polypeptides and polynucleotides encoding the same.

VEGFs are a class of proteins thought to have growth-promoting activities on vascular tissues. Known members of the VEGF family include, but are not limited to, VEGF-A, VEGF-B, VEGF-C, VEGF-D and VEGF-E, any of which may be incorporated into the bone in-growth and on-growth formulations of the embodiments, in addition to polypeptides and polynucleotides encoding the same.

TGF-bs are a class of proteins thought to have growth-promoting activities on a range of tissues, including connective tissues. Known members of the TGF-b family include, but are not limited to, TGF-b-1, TGF-b-2, and TGF-b-3, any of which may be incorporated into the bone in-growth and on-growth formulations of the embodiments, in addition to polypeptides and polynucleotides encoding the same.

Known GDFs include, but are not limited to, GDF-1, GDF-2, GDF-3, GDF-7, GDF-10, GDF-11, and GDF-15. GDF-1 polynucleotides and polypeptides correspond to GenBank Accession Numbers M62302, AAA58501, and AAB94786; GDF-2 polynucleotides and polypeptides correspond to GenBank Accession Numbers BC069643, BC074921, Q9UK05, AAH69643, and AAH74921; GDF-3 polynucleotides and polypeptides correspond to GenBank Accession Numbers AF263538, BC030959, AAF91389, AAQ89234, and Q9NR23; GDF-7 polynucleotides and polypeptides correspond to GenBank Accession Numbers AB158468, AF522369, AAP97720, and Q7Z4P5; GDF-10 polynucleotides and polypeptides correspond to GenBank Accession Numbers BC028237 and AAH28237; GDF-11 polynucleotides and polypeptides correspond to GenBank Accession Numbers AF100907, NP_{—}005802 and O95390; and GDF-15 polynucleotides and polypeptides correspond to GenBank Accession Numbers BC008962, BC000529, AAH00529, and NP_{—}004855.

Known CDMPs and LMPs include, but are not limited to, CDMP-1, CDMP-2, LMP-1, LMP-2, and LMP-3. CDMP-1 polynucleotides and polypeptides correspond to GenBank Accession Numbers NM_{—}000557, U13660, NP_{—}000548 and P43026; CDMP-2 polypeptides correspond to GenBank Accession Numbers and P55106; LMP-1 polynucleotides and polypeptides correspond to GenBank Accession Numbers AF345904 and AAK30567; LMP-2 polynucleotides and polypeptides correspond to GenBank Accession Numbers AF345905 and AAK30568; and LMP-3 polynucleotides and polypeptides correspond to GenBank Accession Numbers AF345906 and AAK30569.

Other osteoinductive and osteoconductive factors, agents, and compounds such as hydroxyapatite (HA), tricalcium phosphate (TCP), collagen, fibronectin (FN), osteonectin (ON), endothelial cell growth factor (ECGF), cementum attachment extracts (CAE), ketanserin, human growth hormone (HGH), animal growth hormones, epidermal growth factor (EGF), interleukin-1 (IL-1), human alpha thrombin, insulin-like growth factor (IGF-1), platelet derived growth factors (PDGF), and fibroblast growth factors (FGF, bFGF, etc.) also may be included in the coating compositions described herein.

Some of the coating compositions described herein may include polypeptide compositions, which may be delivered by gene therapy vectors harboring the polynucleotides encoding the polypeptide of interest. The vector may be, for example, a phage, plasmid, viral, or retroviral vector. The gene therapy vectors may be included only in portions of the coating where tissue attachment is desired.

Gene therapy methods require a polynucleotide which codes for the desired polypeptide and any other genetic elements necessary for the expression of the polypeptide by the target tissue. Such gene therapy and delivery techniques are known in the art. See, for example, International Publication No. WO 90/11092, which is herein incorporated by reference. Gene therapy vectors further comprise suitable adenoviral vectors including, but not limited to, those described in Kozarsky and Wilson, Curr. Opin. Genet. Devel., 3:499-503 (1993); Rosenfeld et al., Cell, 68:143-155 (1992); Engelhardt et al., Human Genet. Ther., 4:759-769 (1993); Yang et al., Nature Genet., 7:362-369 (1994); Wilson et al., Nature, 365:691-692 (1993); and U.S. Pat. No. 5,652,224; all of which are herein incorporated by reference.

Suitable gene therapy vectors include gene therapy vectors that do not integrate into the host genome and gene therapy vectors that integrate into the host genome.

A desired polynucleotide also may be delivered in plasmid formulations. Plasmid DNA or RNA formulations refer to polynucleotide sequences encoding osteoinductive polypeptides that are free from any delivery vehicle that acts to assist, promote, or facilitate entry into the cell, including viral sequences, viral particles, liposome formulations, lipofectin or precipitating agents and the like.

Bone growth-promoting agent polypeptides also may be available as heterodimers or homodimers, as well as multimers or combinations thereof. Recombinantly expressed proteins may be in native forms, truncated analogs, muteins, fusion proteins (e.g., fusion proteins with the FC portion of human IgG), and other constructed forms capable of inducing bone, cartilage, or other types of tissue formation as demonstrated by in vitro and ex vivo bioassays and in vivo implantation in mammals, including humans. Examples of preferred fusion proteins include, but are not limited to, ligand fusions between mature osteoinductive polypeptides and the FC portion of human Immunoglobulin G (IgG). Methods of making fusion proteins and constructs encoding the same are well known in the art.

Polypeptide compositions useful in the coating compositions include, but are not limited to, full length proteins, fragments, and variants thereof. In a preferred embodiment, polypeptide fragments are pro-peptide forms of the isolated full length polypeptides. In a particularly preferred embodiment, polypeptide fragments are mature forms of the isolated full length polypeptides. Also preferred are the polynucleotides encoding the propeptide and mature polypeptides of these agents. Preferred embodiments of variant growth-promoting agents include, but are not limited to, full length proteins or fragments thereof that are conjugated to polyethylene glycol (PEG) moieties to increase their half-life in vivo (also known as pegylation). Methods of pegylating polypeptides are well known in the art (see, e.g., U.S. Pat. No. 6,552,170 and European Patent No. 0,401,384 as examples of methods of generating pegylated polypeptides). Embodiments further contemplate the use of polynucleotides and polypeptides having at least 95% homology, more preferably 97%, and even more preferably 99% homology to the isolated bone in-growth and on-growth agent polynucleotides and polypeptides provided herein.

Other compounds that may be included in the bone growth-promoting formulations include platelet derived growth factor (PDGF); insulin-related growth factor-I (IGF-I); insulin-related growth factor-II (IGF-II); fibroblast growth factor (FGF); beta-2-microglobulin (BDGF II); biocidal/biostatic sugars such as dextran and glucose; peptides; nucleic acid and amino acid sequences such as leptin antagonists, leptin receptor antagonists, and antisense leptin nucleic acids; vitamins; inorganic elements; co-factors for protein synthesis; hormones; endocrine tissue or tissue fragments; synthesizers; enzymes such as collagenase, peptidases, and oxidases; polymer cell scaffolds with parenchymal cells; angiogenic agents; antigenic agents; cytoskeletal agents; cartilage fragments; living cells such as chondrocytes, bone marrow cells, mesenchymal stem cells, natural extracts, genetically engineered living cells, or otherwise modified living cells; autogenous tissues such as blood, serum, soft tissue, and bone marrow; bioadhesives; periodontal ligament chemotactic factor (PDLGF); somatotropin; antitumor agents and chemotherapeutics such as cis-platinum, ifosfamide, methotrexate, and doxorubicin hydrochloride; immuno-suppressants; permeation enhancers such as fatty acid esters including laureate, myristate, and stearate monoesters of polyethylene glycol; bisphosphonates such as alendronate, clodronate, etidronate, ibandronate, (3-amino-1-hydroxypropylidene)-1,1-bisphosphonate (APD), dichloromethylene bisphosphonate, aminobisphosphonatezolendronate, and pamidronate; pain killers and anti-inflammatories such as non-steroidal anti-inflammatory drugs (NSAID) like ketorolac tromethamine, lidocaine hydrochloride, bipivacaine hydrochloride, and ibuprofen; and salts such as strontium salt, fluoride salt, magnesium salt, and sodium salt.

In addition to osteoinductive proteins discussed above, osteoconductive factors may aid in bone formation (see U.S. Pat. No. 5,707,962). One experienced in the art realizes that osteoconductive factors are those that create a favorable environment for new bone growth, most commonly by providing a scaffold for bone ingrowth. The clearest example of an osteoconductive factor is the extracellular matrix protein, collagen. Other factors that can be considered osteoconductive include nutrients, anti-microbial and anti-inflammatory agents, as well as blood-clotting factors. In addition to these factors, reducing bone absorption by inhibiting osteoclast activity with bisphosphonate also may aid in implant success (see U.S. Pat. No. 5,733,564).

Non-synthetic matrix proteins like collagen, glycosaminoglycans, and hyaluronic acid, which are enzymatically digested in the body, also have been used to deliver BMPs to bone areas (see U.S. Pat. Nos. 4,394,320; 4,472,840; 5,366,509; 5,606,019; 5,645,591; and 5,683,459). In human bone, collagen serves as the natural carrier for BMPs and as an osteoconductive scaffold for bone formation. Demineralized bone in which the main components are collagen and BMPs has been used successfully as a bone graft material (see U.S. Pat. No. 5,236,456). The natural, or synthetic, polymer matrix systems described herein are moldable and release BMPs in the required fashion; however, used alone these polymers serve only as a scaffold for new bone formation. For example, U.S. Pat. Nos. 5,683,459 and 5,366,509 describe an apparatus, useful for bone graft substitute, composed of BMPs injected into a porous polylactide and hyaluronic acid meshwork. Furthermore, an osteogenic device capable of inducing endochondral bone formation when implanted in the mammalian body has been disclosed (see U.S. Pat. No. 5,645,591); this device is composed of an osteogenic protein dispersed within a porous collagen and glycosaminoglycan matrix. These types of devices were designed as an alternative bone graft material to replace the more invasive autograft procedures currently used.

The particular chemical make-up and number of layers can be varied by those skilled in the art depending on the type of intervertebral prosthesis component, the patient, and the disc to be replaced. Preferably, there are two layers, an inner layer positioned adjacent the outer bone-contacting surface of the intervertebral prosthesis component, and an outer layer adjacent the inner layer. It is preferred that the outer layer be a porous compliant layer as described in the embodiments above, and the inner layer be comprised at least in part of the bone growth-promoting material. Designing the coating in this manner facilitates excellent adhesion to the prosthesis component by virtue of the relatively non-porous bone growth-promoting material coating, and excellent adhesion between the inner and outer coating layers since similar carriers and adjuvants can be used to formulate both layers.

The thickness of the compliant coating(s) (coating is referred to herein as including one, two, three, or more coating layers) also will vary depending on the anatomy of the patient, the disc to be replaced, and the particular prosthesis. Preferably, the compliant coating is from about 0.5% to about 30% of the overall thickness of the prosthesis, more preferably from about 1% to about 20% and most preferably from about 1% to about 15% of the overall thickness of the prosthesis prior to application of the coating. Compliant coatings therefore can be designed of a suitable thickness to provide a snug fit upon inserting the prosthesis component into position, thereby facilitating bone growth between the component and the bone.

The compliant coatings can have variable compliance and/or thickness throughout the cross-section, and across the diameter of the prosthesis component by varying the concentrations of materials used to fabricate the coatings, by addition of “softer” (e.g., hydrogel, or more elastic) materials in certain areas and “harder” (e.g., resin materials or more inelastic) materials in other areas. A skilled artisan will appreciate the particular morphology of the disc space to be treated upon reviewing the anatomy of the patient, and consequently, can determine the areas requiring greater or lesser stress (or areas providing greater or lesser load bearing) for the prosthesis component. In accordance with this morphology, the compliant coating compositions can be designed so that they are thicker and/or contain a higher concentration of “harder” components in areas that will be responsible for greater load bearing. Using the guidelines provided herein, a person skilled in the art will be capable of designing a suitable compliant coating based on any of the factors described above, as well as additional factors known to the skilled artisan.

The intervertebral disc prosthesis and/or prosthesis component having a compliant coating applied thereto can be made using techniques known in the art, coupled with the guidelines provided herein. The prosthesis and/or components first are prepared as described in one or more of the documents described previously, and incorporated by reference herein. At least one bone-contacting surface of the prosthesis (i.e., a surface of the prosthesis that is intended to ultimately contact a bony surface) then is coated with a compliant coating. Prior to coating, the bone-contacting surface may be pre-treated with an etching solution, or by a surface roughening technique, as is well known in the art.

Coating of the bone-contacting surface can be accomplished using a variety of coating techniques. Preferably, the bone-contacting surface first is coated with a relatively (preferably substantially or totally) non-porous inner layer. The inner layer may contain at least one bone growth promoting agent, as well as any of the aforementioned materials (immunosuppressive agents, antiviral agents, antibiotics, etc.), in addition to conventional coating layer carriers and adjuvants. The inner layer then preferably is coated with a compliant layer, optionally having a variable thickness and concentration across its cross-section. Coating can be effected using any known coating technique, including but not limited to, spray coating, extrusion coating, plasma sputtering, chemical vapor deposition, injection of materials and curing using energy (light, heat, moisture, etc.), growth of organic layer, and the like. Alternatively, the bone-contacting surface can be coated with one compliant layer comprising at least one bone growth promoting agent, as opposed to two separate layers.

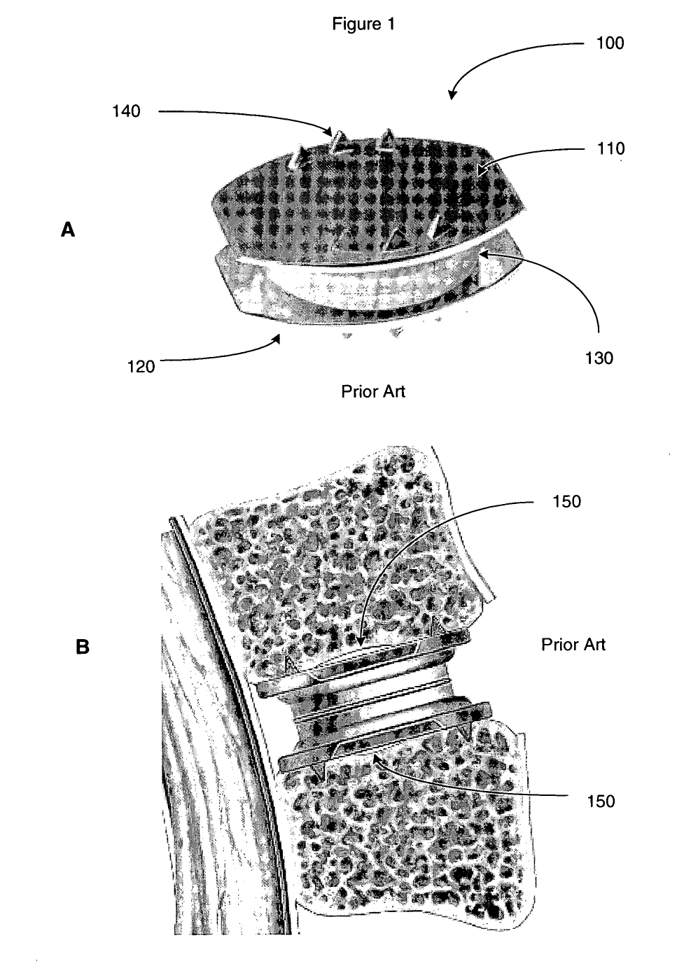

The figures appended hereto are intended to illustrate exemplary embodiments and to explain in more detail the benefits and advantages of the embodiments. FIG. 1 illustrates a known intervertebral disc prosthesis—the CHARITÉ™ Artificial Disc—at present, the only commercially available disc prosthesis, commercially available from DePuy Spine, Raynham, Massachusetts. The prosthesis 100 of FIG. 1, embodiment A, includes an upper endplate 110, lower endplate 120 and central member 130. The prosthesis 100 further includes bone contacting points 140, or ridges to securely attach the prosthesis 100 to the adjacent vertebral bodies.

As shown in FIG. 1, embodiment B, when inserted between vertebral bodies, the prosthesis does not conform well to the morphology of the bony surface of each vertebral body endplate. This is so even though prosthesis 100 could be coated with a bone growth promoting composition. As shown, gaps 150 exist between the bony endplate surface of each vertebral body and the prosthesis 100. These gaps create poor contact between the prosthesis and the bone, which results in poor bony ingrowth between the prosthetic endplate and the vertebral body endplate. Thus, such a prosthesis could ultimately fail to seat properly in the disc space and partially dislodge causing extreme pain, as well as other life threatening injuries.

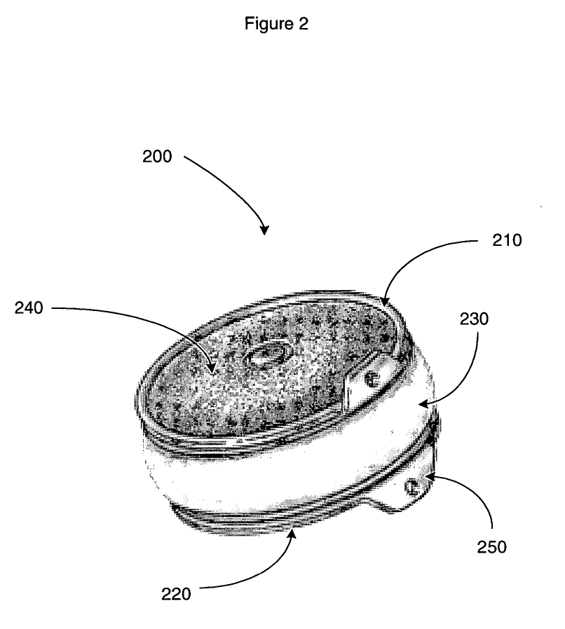

FIG. 2 illustrates an embodiment whereby an intervertebral disc prosthesis 200 is provided with a compliant coating 240. The intervertebral disc prosthesis 200 includes upper endplate 210, lower endplate 220, polymeric, flexible central portion 230, and vertebral attachment portions 250. While FIG. 2 illustrates a compliant coating 240 only on upper undplate 210, the compliant coating also may be applied to lower endplate 220, as well as vertebral attachment portions 250.

Without the compliant coating 240 on intervertebral disc prosthesis 200, a surgeon would typically have to prepare the upper and lower vertebral body endplates to conform to the shape of the upper endplate 210 and lower endplate 220, respectively. Such preparation of the endplates is described in, for example, U.S. Pat. Nos. 6,083,228; 6,517,544; and 6,537,279; and U.S. patent application Publication Nos.: 2002/0035400; 2002/0128715; 2002/0151901; 2003/0187448; 2003/0130662; and 2005/0015091. Use of compliant coating 240 on one or more bone-contacting surfaces of intervertebral disc prosthesis 200 permits introduction of the prosthesis into the disc space, without having to prepare the vertebral body endplates, as described above, or at least without having to mill them to the extent that would be required without the coating 240.

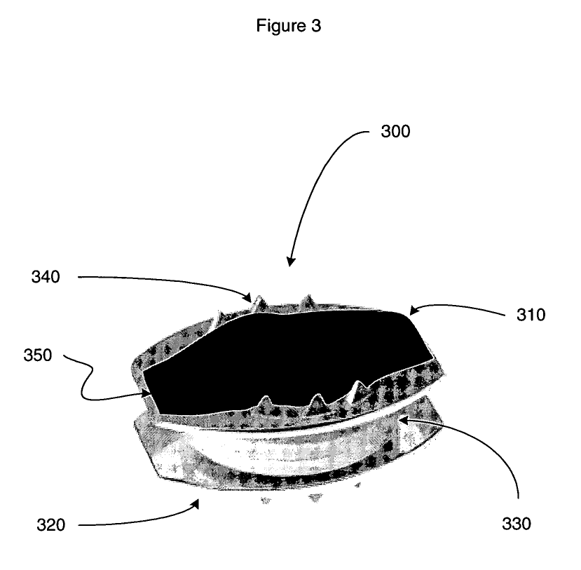

FIG. 3 illustrates another embodiment whereby the intervertebral prosthesis of FIG. 1, now prosthesis 300, is coated with a compliant coating 350. Intervertebral prosthesis 300 includes upper endplate 310, lower endplate 320, and central member 330. The prosthesis 300 further includes bone contacting points 340, or ridges to securely attach the prosthesis 300 to the adjacent vertebral bodies. Unlike the commercially available prosthesis of FIG. 1, however, prosthesis 300 of the embodiments described herein includes a compliant coating 350 that will fill in the gaps 150 (see, FIG. 1) and provide contact between the bony endplates of the adjacent vertebral bodies, and the upper and lower endplates 310, 320, respectively, of prosthesis 300. This contact, coupled with the optional and preferred presence of bone growth promoting substances in the coating 350 (e.g., either in the upper preferably porous layer, or present in one or more inner not-as-porous layers closer to the prosthetic endplates), will facilitate improved bone in-growth, when compared to the prosthesis 100 of FIG. 1 that does not include a compliant coating.

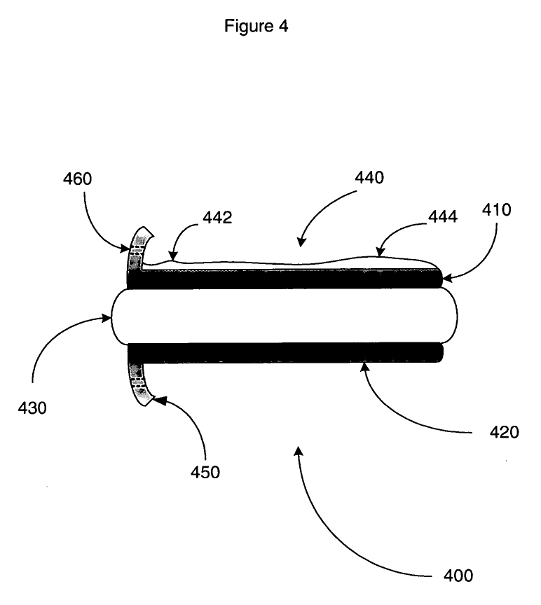

FIG. 4 is a side perspective view of an intervertebral prosthesis 400, with a compliant coating 440 having variable thickness and chemical make-up throughout its cross-section and/or across the length of the prosthesis 400. Again, although the prosthesis 400 is shown with coating 440 only on one bone-contacting surface (endplate 410), skilled artisans will appreciate that a compliant coating also could and preferably should be applied to endplate 420, as well as bone attachment flange 450. The intervertebral prosthesis 400 includes a flexible central member 430, and bone attachment flanges 450, the flanges each including at least one through-hole 460 for a vertebral body bone anchor to anchor the prosthesis to the adjacent vertebral bodies.

As shown in FIG. 4, compliant coating 440 has variable thickness and can have a variable chemical make-up. For example, a skilled artisan might conclude from assessing the patient's vertebral body morphology that high stress and load bearing areas for prosthesis 400 will exist at areas 442 and 444. These areas may correspond, for example, to high points in the vertebral body endplates. Surgeons skilled in the art typically know where the high load areas are in the endplates of the vertebral bodies. For example, these high points usually exist around the periphery (epophesial ring) where the hardest bone of the endplate exists, which usually is where the highest strength is required. The center of the endplates tend to be the weakest, softest, and most vascular area. Accordingly, the compliant coating 440 can be thicker in those regions where the endplates are the thickest to provide better load bearing capabilities, or the coating may contain “harder” or “softer” components in these areas.

In addition to higher load bearing areas across the cross-section of the intervertebral disc prosthesis 400, the skilled artisan may recognize greater undulations in the vertebral body endplate morphology in certain areas, and consequently, desire a compliant coating with a greater thickness in these areas. The coating also may be more compliant (e.g., comprise a greater concentration of more flexible components) in the thicker areas to permit insertion into the disc space. This would provide greater flexibility in the thicker areas that need to flex more during insertion of the intervertebral disc prosthesis. In addition, the coating may include flowable or expandable materials in the areas requiring a thicker coating. Application of energy (light, heat, insertion or fluids or contact with natural body fluids, etc.) then can render the material flowable or can expand the materials present in these areas to thicken the coating and provide better bony contact.

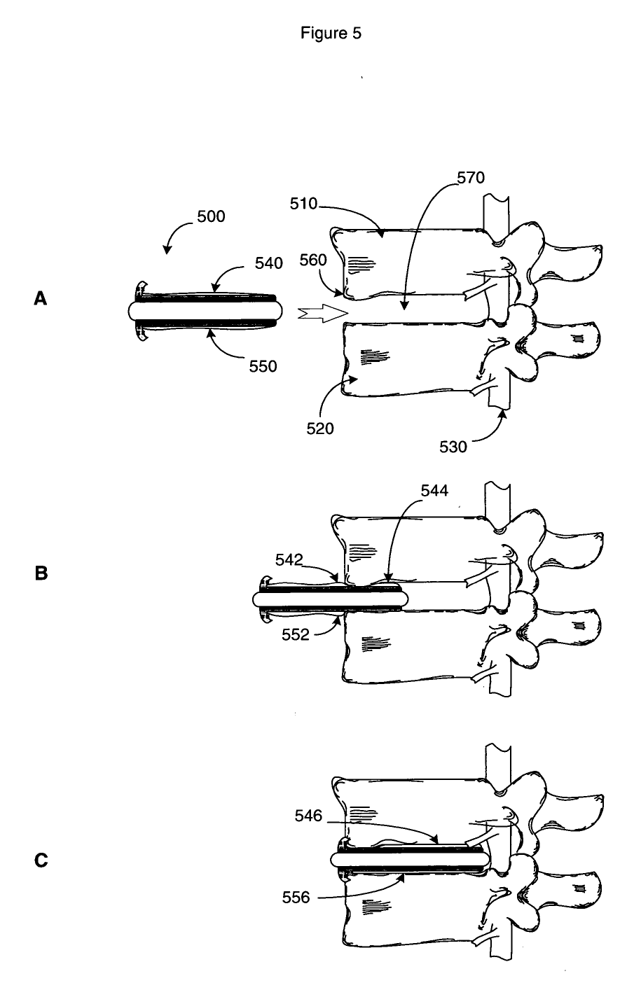

FIG. 5 illustrates an exemplary method of inserting an intervertebral disc prosthesis 500. Prior to insertion of the intervertebral disc prosthesis 500, a surgeon would access the disc space 570 using techniques known in the art.

Embodiments of the invention are particularly suitable for minimally invasive surgical techniques. Accessing disc space 570 may be from any approach to the spine, and typically involves use of a guidewire to pinpoint the entry point, followed by successive dilation to provide a delivery channel or port suitable for insertion of instruments to perform the requisite surgical procedures. The annulus fibrosus typically is removed in whole or in part, followed by partial or complete evacuation of the intervertebral disc space 570.

Prior to the present invention, if one were to insert an intervertebral disc prosthesis similar to that shown in FIG. 5, without the compliant coating, the surgeon would need to prepare the bony surfaces of upper vertebral body 510 and lower vertebral body 520 using a milling or drilling apparatus. Excess bony endplate material 560 also typically would be removed, and the vertebral body endplate surfaces adjacent disc space 570 would be milled to a shape complementary that of the prosthesis.

Milling is an extremely dangerous procedure, with extra precautions required to avoid damage to spinal cord 530.

The present inventors have discovered that the compliant coating 540, 550 now present on the intervertebral disc prosthesis 500 permits insertion of prosthesis 500 without excessive milling of the vertebral body endplate surfaces adjacent disc space 570, and preferably without any milling. Those skilled in the art will appreciate that the morphology of some patients may require some minor milling to remove small portions of the vertebral body endplate surfaces adjacent disc space 570 if, for example, the undulations of these surfaces across the disc space were greater in dimension than the maximum thickness of the compliant coating 540, 550, combined with any undulations present on the surface of the prosthesis 500.

FIG. 5, embodiment A illustrates positioning intervertebral disc prosthesis 500 in the appropriate orientation for insertion, and then advancing prosthesis 500 in the direction of the arrow into the disc space. FIG. 5, embodiment B illustrates the prosthesis 500 as it has advanced partially into the disc space. Here, one can readily see compliant coatings 540, 550 deforming to allow prosthesis to advance past the edge endplate 560 of vertebral body 510. For example, compliant coating 540 is pinched at this point producing a thicker portion 544 inside the disc space and a thicker portion 542 outside the disc space. At the same time, compliant coating 550, is pinched to produce a thicker portion 552 outside the disc space.

Similar deformation of compliant coatings 540, 550 will take place as the prosthesis 500 is advanced fully into disc space 570.

FIG. 5, embodiment C illustrates intervertebral disc prosthesis 500 after insertion into disc space 570. Compliant coatings 540, 550 have sufficiently deformed and essentially molded to the geometry and morphology of the vertebral body endplate surfaces adjacent disc space 570. In their implanted configuration, compliant coatings 546, 556, provide superior contact with the vertebral body endplate surfaces adjacent disc space 570, which enables better bony in-growth, and thus, better fusion of the endplates of intervertebral disc prosthesis 500. This friction fit also provides a better initial fit, and provides for enhanced load bearing capabilities across the cross-section of the prosthesis 500.

Those skilled in the art will appreciate that a compliant coating may be present on any bone-contacting surface of any component of an intervertebral disc prosthesis.

These surfaces may be present on plates, screws, flanges, endplates, bags, outer sheaths, etc. The compliant coating may be comprised of a single layer or multiple layers, and preferably is comprised of at least two layers: (i) an inner relatively or substantially non-porous bone-growth promoting layer; and (ii) an outer compliant layer. The compliant coating may resemble a sponge-like material (e.g., collagen, polyurethanes, etc.), a tissue scaffolding material, or a resilient coated layer.

The foregoing detailed description is provided to describe the invention in detail, and is not intended to limit the invention. Those skilled in the art will appreciate that various modifications may be made to the invention without departing significantly from the spirit and scope thereof.

What is claimed is:

1. An intervertebral disc prosthesis comprising a component having a compliant coating on at least one surface, the at least one surface intended to contact a vertebral bone in a mammal's body, the compliant coating at least partially conforming to the vertebral bone.

2. The intervertebral disc prosthesis as claimed in claim 1, wherein the prosthesis is capable of implantation into an at least partially evacuated disc space without excessive vertebral body milling to conform the bone surface to fit the prosthesis.

3. The intervertebral disc prosthesis as claimed in claim 1, wherein the compliant coating includes two layers, an inner layer positioned adjacent the at least one surface, and an outer layer positioned adjacent the inner layer, the outer layer having a pore size within the range of from about 100 to about 500 microns.

4. The intervertebral disc prosthesis as claimed in claim 3, wherein the inner layer is a relatively non-porous, non-compliant layer, and the outer layer is a compliant porous layer.

5. The intervertebral disc prosthesis as claimed in claim 1, wherein the compliant coating is comprised of a material selected from the group consisting of resorbable fibers, semi-resorbable fibers; woven or interconnected fibers, metallic intepenetrating network fibers, polymeric intepenetrating network fibers, three-dimensional polyethylene, polyesters, PEEK films, titanium mesh fibers, nitinol fibers, PET, PTFE fibers, graphite fibers, polysulfones, hydrogels, flexible tissue scaffolding, resilient film-forming olefinic resins, biologically grown materials, and combinations thereof.

6. The intervertebral disc prosthesis as claimed in claim 1, wherein the compliant coating comprises at least one bone growth promoting agent.

7. The intervertebral disc prosthesis as claimed in claim 5, wherein the inner layer comprises at least one bone growth promoting agent.

8. The intervertebral disc prosthesis as claimed in claim 1, wherein the component is a prosthesis endplate.

9. The intervertebral disc prosthesis as claimed in claim 1, wherein the component is a vertebral attachment flange.

10. The intervertebral disc prosthesis as claimed in claim 1, wherein the prosthesis comprises an upper prosthesis endplate, a lower prosthesis endplate, a flexible central member positioned between the upper and lower prosthesis endplates, and a means for attaching the upper prosthesis endplate and/or lower prosthesis endplate to a vertebral body bony surface.

11. The intervertebral disc prosthesis as claimed in claim 10, wherein the means for attaching include vertebral attachment flanges and a vertebral bone anchor.

12. The intervertebral disc prosthesis as claimed in claim 10, wherein the means for attaching include at last one contact point or ridge.

13. The intervertebral disc prosthesis as claimed in claim 1, wherein the thickness and/or concentration of components of the compliant coating vary across the cross-section of the intervertebral disc prosthesis.

14. The intervertebral disc prosthesis as claimed in claim 1, wherein the compliant coating has a thickness within the range of from about 0.5% to about 30% of the overall thickness of the prosthesis.

15. A method of making an intervertebral disc prosthesis and/or component thereof comprising forming a disc prosthesis and/or component thereof, and coating at least one bone-contacting surface of the prosthesis and/or component thereof with a coating material that, when coated on the surface, provides a compliant surface capable of deformation and partial conformation to a vertebral bone in a mammal's body.

16. The method as claimed in claim 15, wherein coating comprises forming an inner layer adjacent the bone-contacting surface, and forming an outer layer adjacent the inner layer.

17. The method as claimed in claim 16, wherein the inner layer is a relatively non-porous, non-compliant layer, and the outer layer is a compliant porous layer.

18. A method of inserting an intervertebral disc prosthesis into an at least partially evacuated disc space comprising:
partially evacuating the disc space positioned between two vertebral bodies, each vertebral body having a vertebral body bony surface adjacent the disc space;
providing an intervertebral disc prosthesis having at least one component with a compliant coating on at least one surface, the at least one surface intended to contact and at least partially conform to a vertebral bone in a mammal's body; and
inserting the prosthesis into the at least partially evacuated disc space without excessive modification of at least one of the vertebral body bony surface prior to insertion.

19. The method as claimed in claim 18, wherein the prosthesis is inserted without any modification of the at least one vertebral body bony surface adjacent the disc space.

20. The method as claimed in claim 18, wherein the compliant coating includes two layers, an inner layer positioned adjacent the at least one surface, and an outer layer positioned adjacent the inner layer.

21. The method as claimed in claim 20, wherein the inner layer is a relatively non-porous, non-compliant layer, and the outer layer is a compliant porous layer.

22. The method as claimed in claim 18, wherein the compliant coating is comprised of a material selected from the group consisting of resorbable fibers, woven or interconnected fibers, metallic intepenetrating network fibers, polymeric intepenetrating network fibers, three-dimensional polyethylene, polyesters, PEEK films, titanium mesh fibers, nitinol fibers, PET, PTFE fibers, graphite fibers, polysulfones, hydrogels, flexible tissue scaffolding, resilient film-forming olefinic resins, biologically grown materials, and combinations thereof.

23. The method as claimed in claim 18, wherein the compliant coating comprises at least one bone growth promoting agent.

24. The method as claimed in claim 20, wherein the inner layer comprises at least one bone growth promoting agent.

25. The method as claimed in claim 18, wherein the component is a prosthesis endplate.

26. The method as claimed in claim 18, wherein the component is a vertebral attachment flange.

27. The method as claimed in claim 18, wherein the prosthesis comprises an upper prosthesis endplate, a lower prosthesis endplate, a flexible central member positioned between the upper and lower prosthesis endplates, and a means for attaching the upper prosthesis endplate and/or lower prosthesis endplate to a vertebral body bony surface.

28. The method as claimed in claim 27, wherein the means for attaching include vertebral attachment flanges and a vertebral bone anchor.

29. The method as claimed in claim 27, wherein the means for attaching include at last one contact point or ridge.

30. The method as claimed in claim 18, wherein the thickness and/or concentration of components of the compliant coating vary across the cross-section of the intervertebral disc prosthesis.

31. The method as claimed in claim 18, wherein the compliant coating has a thickness within the range of from about 0.5% to about 30% of the overall thickness of the prosthesis.
